(19) United States
(12) Patent Application Publication
(10) Pub. No.: US 2006/0178595 A1
(43) Pub. Date: Aug. 10, 2006

(54) System and method for measuring pain impact

(76) Inventors: Stefaan Schatteman, Sint-Niklaas (BE); Kris Schellens, Lochristi (BE)

Correspondence Address: DUANE MORRIS LLP;PATENT DEPARTMENT, 380 LEXINGTON AVENUE, NEW YORK, NY, 10168-0002, US

(21) Appl. No.: 11/300,562

(22) Filed: Dec. 13, 2005

(30) Foreign Application Priority Data
Dec. 13, 2004 (EP) 000265913-0001
Dec. 13, 2004 (EP) 000265913-0002

(51) Int. Cl. A61B 19/00

(52) U.S. Cl. 600/553

(57) ABSTRACT

The present invention relates generally to a system and method useful for monitoring the effectiveness of a pain treatment. Specifically, it relates to a system and method for measuring and recording pain intensity and/or pain impact as perceived by a subject. In specific embodiments, the system and method utilize multiple scales in conjunction with a pain treatment program to provide information regarding pain treatment effectiveness based on recording of pain intensity and pain impact experienced by a subject. The invention further relates to a device for measuring and recording the intensity of pain and the level of pain impact as perceived by a subject.

This application claims the benefit of European Community Design Registration Nos. 000265913-0001, and 000265913-0002, registered on Dec. 13, 2004. The disclosure of the European Community Design Registrations being incorporated by reference herein in its entirety.

### 1. FIELD OF THE INVENTION

The present invention relates generally to a system and method useful for monitoring the effectiveness of a pain treatment. Specifically, it relates to a system and method for measuring and recording the level of pain and/or pain impact perceived by a subject. In specific embodiments, the system and method utilize multiple scales in conjunction with a pain treatment program to provide information regarding pain treatment effectiveness based on recording of the level of pain and pain impact experienced by a subject.

### 2. BACKGROUND OF THE INVENTION

The measurement of pain and pain relief, and therefore quantifying treatment effectiveness, poses significant difficulties. Healthcare providers are frequently faced with the problem of diagnosing and treating patients suffering from varying levels of pain. The difficulty in properly diagnosing and treating the varying levels of pain often results from the patients' inability to accurately describe the pain that they are experiencing and how that pain affects their well-being.

Accordingly, there is a need for a more convenient system and method for measuring and recording the level of pain and/or pain impact on a subject over time or during the course of treatment of pain, or treatment of disease or condition that causes the pain. Furthermore, there is a need for a system and method to more accurately record, assess and measure the intensity of pain and its impact on a patient so that pain intensity and/or pain impact can be compared over time or during the course of a treatment.

### 3. SUMMARY OF THE INVENTION

The present invention relates to a system and method for measuring pain impact and/or pain intensity. In specific embodiments, the present invention relates to a system and method for allowing a healthcare provider to measure changes in pain impact and/or pain intensity over a period of time or during the course of a treatment to determine overall effectiveness of a pain treatment. In specific embodiments, when the patient periodically visits the healthcare provider, the pain impact and/or pain intensity are measured. The healthcare provider then collects a set of data at a particular point in time for reference and comparison with a set of data collected at another point in time. The change in pain impact and/or pain intensity are measured and compared over time. The present invention provides information necessary for establishing and validating the effectiveness of the pain treatment.

In one embodiment, the present invention relates to a system and method for monitoring the effectiveness of a treatment. In one specific embodiment, the present invention relates to a system and method for monitoring the effectiveness of a treatment of pain. In another specific embodiment, the present invention relates to a system and method for monitoring the effectiveness of treatment of a disease or condition that causes pain. The present invention relates to a system and method for measuring and recording pain impact and/or pain intensity in conjunction with a pain, disease or condition treatment program to provide information regarding pain treatment effectiveness based upon the recording of perceived levels of pain impact and/or pain experienced by a patient.

The present invention is directed to a device for measuring the level of pain impact and/or pain intensity of a subject comprising: (a) a substrate comprising a plurality of scales wherein each of the scales comprises a first end and a second end and wherein the plurality of scales are connected at the first end of each scale; (b) an indicator for each of the scales that is capable of moving or being located between the first end and the second end of each scale; (c) the indicators on each pair of adjacent scales are connected or connectable to a line; (d) the line and the scales enclose a first surface area of a first size.

In a specific embodiment, the plurality of scales comprises a first scale, a second scale, a third scale and a fourth scale. In a specific embodiment, the first end of the first scale has a minimum value and the second end of the first scale has a maximum value. In a specific embodiment, the first scale comprises a spectrum of color shades which vary from the first end to the second end. In a specific embodiment, the first scale measures the intensity of pain. In a specific embodiment, the second scale measures the level of pain impact on quality of life. In a specific embodiment, the third scale measures the level of pain impact on work abilities. In a specific embodiment, the fourth scale measures the level of pain impact on daily activities. In a specific embodiment, the first scale comprises graduated intervals. In a specific embodiment, the substrate further comprises a fifth scale.

The present invention is also directed to a method of measuring the level of pain impact comprising the steps of: (a) providing a substrate comprising a plurality of scales, each scale comprising a first end and a second end with the first ends of the scales meeting; (b) marking a first point at a position along each scale; and (c) connecting each pair of points on adjacent scales with a first line in which the first lines and the scales enclose a first surface area having a first size. The present invention is also directed to a device for measuring the level of pain impact, the device comprising a substrate comprising a plurality of scales, each scale comprising a first end and a second end with the first ends of the scales meeting and an indicator that is capable of moving or being located between the first end and the second end.

### 3.1 DEFINITIONS

As used herein, the term “therapeutic agent” includes biologically active materials, such as pharmaceuticals, drugs, genetic materials, and biological materials.

As used herein, the term “therapeutically effective amount” refers to that amount of the therapeutic agent sufficient to treat or ameliorate pain or certain diseases or conditions. A therapeutically effective amount may refer to that amount of therapeutic agent sufficient to delay or minimize pain associated with certain diseases or conditions. A therapeutically effective amount may also refer to that amount of the therapeutic agent that provides a therapeutic benefit in the treatment or management of certain diseases or conditions that give rise to pain.

As used herein, the terms “subject” and “patient” are used interchangeably. As used herein, a subject is a human.

### 4. FIGURES

FIG. 1 shows a substrate comprising a plurality of scales useful for an embodiment of the system and method of the present invention.

FIG. 2 shows a substrate comprising a plurality of scales useful for an embodiment of the system and method of the present invention.

FIG. 3 shows a substrate comprising four scales useful for an embodiment of the system and method of the present invention. One of the scales is marked at various intervals from a minimum value of zero to a maximum value of 10.

FIG. 4 shows a substrate comprising four scales and a table for recording a score from each scale for measuring: (i) the intensity of pain; (ii) impact of the pain on work; (iii) impact of the pain on daily activities; and (iv) impact of the pain on quality of life, during the course of a first, second and third assessment.

FIG. 5 shows an embodiment of a device of the present invention.

### 5. DETAILED DESCRIPTION OF ILLUSTRATIVE EMBODIMENTS

For clarity of disclosure, and not by way of limitation, the detailed description of the invention is divided into the subsections which follow.

5.1 Methods and Systems For Measuring and Recording Pain intensity and/or Pain Impact

The present invention provides a system and method to measure and record pain impact and/or pain intensity as perceived by a subject. The system comprises the steps of: (i) providing a substrate comprising a plurality of scales for measuring pain impact and/or intensity of pain as perceived by the subject, each scale comprising a first end and a second end with the first ends meeting; (ii) marking a point at a position along each scale as determined by the subject; and (iii) connecting each pair of points on adjacent scales with a line so that the lines and the scales enclose a surface area on the substrate.

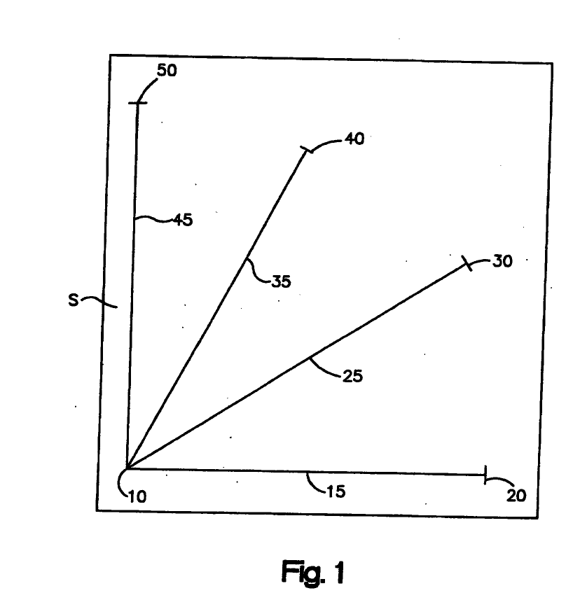

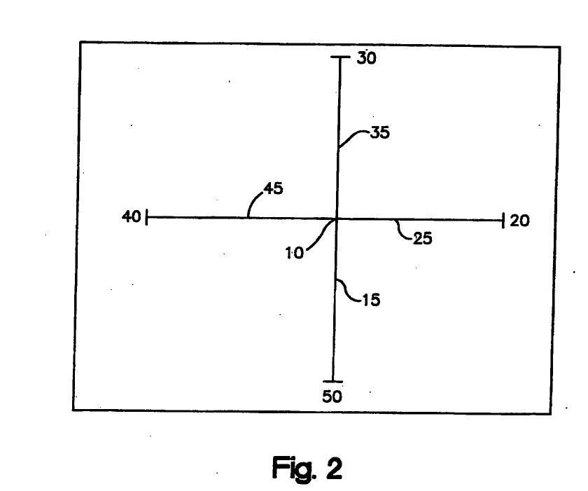

The system and method comprise providing a substrate, such as but not limited to, a chart, a card, a paper, a cork board, a foam board, or a magnetic board, comprising a plurality of scales, or a computer screen on which the chart or plurality of scales is generated by appropriate software. In a specific embodiment, the substrate comprises four scales. FIG. 1 shows a substrate S comprising a first scale 15, a second scale 25, a third scale 35, and a fourth scale 45. Each scale comprises a first end 10 and a second end, 20, 30, 40, and 50, respectively. The first end of each scale converges at a single point 10. The angle between each scale may be of any value. In a specific embodiment, the angle between two adjacent scales is about 30° as shown in FIG. 1. As shown in FIG. 2 as a preferred embodiment, the angle between two adjacent scales is 90°.

FIG. 2 shows a substrate S comprising a first scale 25, a second scale 35, a third scale 45, and a fourth scale 15. Each scale comprises a first end 10 and a second end 20, 30, 40, and 50, respectively. The first end of each scale converges at a single point 10. In a preferred embodiment, the first scale measures the amount or intensity of a pain as perceived by a subject. The subject uses the scales to indicate the level of pain that the subject is experiencing by marking a point at an appropriate position along the scale. In specific embodiments, the first end has a minimum value and the second end has a maximum value. In specific embodiments, the first end has a value of zero and the second end has a value greater than zero. In specific embodiments, the scale is divided into unit measurements. In specific embodiments, the scale is divided evenly into unit measurements of numeric values of ascending order such as 0, 1, 2, 3, 4, 5, 6, 7, 8, 9, 10. In specific embodiments, the scale comprises categorical pain descriptors which may include sensory pain descriptors or emotional pain descriptors. In specific embodiments, the scale comprises categorical pain descriptors that are graphical or color coded. In specific embodiments, the scale may comprise both numeric values and non-numeric descriptors, e.g., words. Exemplary scales that may be used for measuring the intensity of pain are provided in Section 5.1.1.

In specific embodiments, the substrate comprises one or more scales for the measurement of pain impact on a subject. In a preferred embodiment, the substrate comprises one scale for the measurement of pain intensity and three scales for the measurement of pain impact. The impact of pain may affect many aspects of a subject's life, including but not limited to, work, daily activities, and quality of life as discussed in Section 5.1.2.

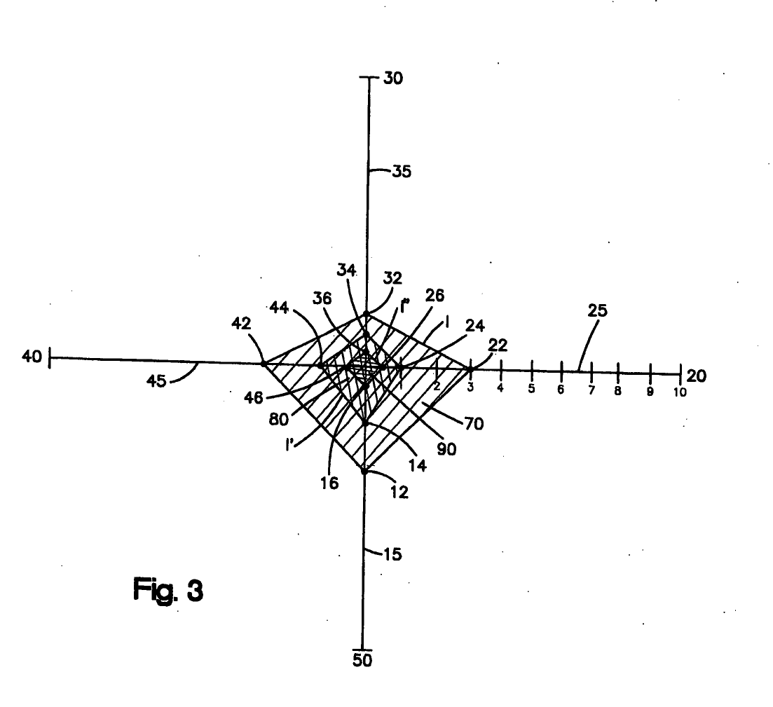

The system and method of the present invention further comprise marking a first point at a position between the first end and the second end inclusively on each of the scales of the substrate. The subject is asked to use a pain impact scale to quantify the extent to which the pain limits his/her activities. FIG. 3 shows that a first point 22 is marked between the first end 10 and the second end 20 on the first scale 25. A second point 32 is marked between the first end 10 and the second end 30 on the second scale 35. A third point 42 is marked between the first end 10 and the second end 40 on the third scale 45. A fourth point 12 is marked between the first end 10 and the second end 50 on the fourth scale 15.

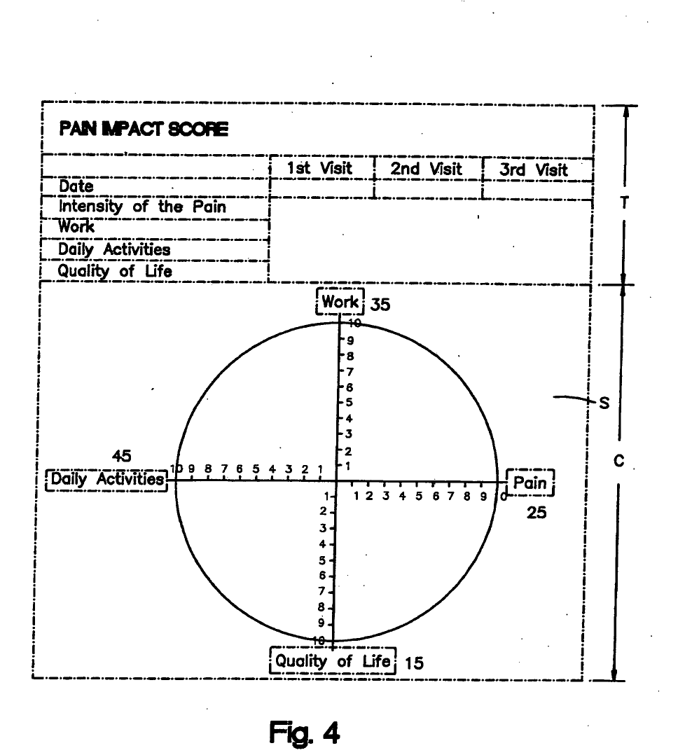

FIG. 4 shows a substrate S useful for the system and method of the present invention. Specifically, a subject is asked to use a first pain intensity scale, 25, to quantify the amount of pain that the subject is experiencing at a time point, a second pain impact scale to quantify the extent to which the pain limits professional activities, 35, a third pain impact scale to quantify the extent to which the pain limits daily activities, 45, a fourth pain impact scale to quantify the extend to which the pain limits quality of life, 15. In a specific embodiment, each of the four scales is evenly marked at various intervals and is graduated with unit measurements from 0 to 10. The measurement is taken at the time of evaluation and recorded as a numeric value in a table (T) and a chart (C) provided on the substrate S. For example, a 0 out of 10 is no influence and a 10 out of 10 is major influence on activities. A pain intensity scale 25 is used for measuring the intensity of pain. For example, a 0 out of 10 is no pain and a 10 out of 10 is maximum pain. A pain impact scale is used for measuring pain intensity 25, pain impact on work, 35, on daily activities, 45, and on quality of life, 15. The subject is asked to use a pain impact scale to quantify the extent to which the pain limits work 35. For example, a 0 out of 10 is no influence and a 10 out of 10 is major influence on work. The subject is asked to use a pain impact scale to quantify the extent to which the pain limits daily activities 45. For example, a 0 out of 10 is no influence and a 10 out of 10 is major influence on daily activities. The subject is asked to use a pain impact scale to quantify the extent to which the pain limits quality of life 15, including but not limited to social activities, quality of sleep, moments of pleasure. For example, a 0 out of 10 is no influence and a 10 out of 10 is major influence on quality of life. In specific embodiments, a point is marked on each scale corresponding to such pain measurements.

The system and method of the present invention further comprise connecting each pair of points on adjacent scales with a first line. In FIG. 3, first points 22, 32, 42, and 12 are connected with a first line, 1, such that the first lines and scales 25, 35, 45, and 15 enclose a first surface area 70 having a first size. The first size represents a first measurement of pain impact at a certain pain intensity at a first time point. In specific embodiments, second points 24, 34, 44, 14 are connected with a second line, 1′, such that the second lines and scales 25, 35, 45, and 15 enclose a second surface area 80 having a second size. The second size represents a second measurement of pain impact at a certain pain intensity at a second time point. In specific embodiments, third points 26, 36, 46, 16 are connected with a third line, 1″, such that the third lines and scales 25, 35, 45, and 15 enclose a third surface area 90 having a third size. The third size represents a third measurement of pain impact at a certain pain intensity at a third time point.

In a specific embodiment, the first time point is a first visit to a health care provider. The second time point is a second visit to a health care provider. The third time point is a third visit to a health care provider, etc. In a specific embodiment, the second time point is after the administration of a first treatment for a period of time. In a specific embodiment, the third time point is after the administration of a second treatment for a period of time. In a specific embodiment, the first time point and the second time point and/or the second time point and the third time point has an interval of at least 1-2 days, at least 2-7 days, at least 1 week-4 weeks, at least 1-2 months, at least 2-6 months, or at least 6 months to one year.

The system and method of the present invention further comprise comparing the first size of the first surface area to the second size of the second surface area wherein a decrease in the second size of the second surface area compared to the first size of the first surface area indicates a decrease in pain intensity and/or pain impact. In specific embodiments, the decrease in pain intensity and/or pain impact indicates an effective treatment method. In specific embodiments, an increase in the second size of the second surface area compared to the first size of the first surface area indicates an increase in pain intensity and/or pain impact. In specific embodiments, the increase in pain intensity and/or pain impact indicates an ineffective treatment method.

5.1.1 Methods of Measuring Pain

The level of pain may be measured by the amount and/or the intensity of pain. Categorical pain descriptors, such as verbal and/or numerical categorical pain descriptors, may be used. Verbal pain descriptors may, for example, include from the first end of a scale, no pain, minimal pain, constant minimal to intermittent slight pain, constant slight pain, constant slight to intermittent moderate pain, constant slight to frequent moderate pain, intermittent moderate pain, frequent moderate pain, constant moderate pain, constant moderate to intermittent severe pain, and constant severe pain at the second end of the scale. Numerical categorical pain descriptors may include from the minimum end of a scale, 0, 1, 2, 3, 4, 5, 6, 7, 8, 9, and 10 at the maximum end of the scale. The categorical pain descriptors may comprise a spectrum of color shades or the like, for example, from the first end of a scale, a lightest shade of a color to the darkest shade of a color at the second end of the scale. Alternatively, the categorical pain descriptor may be a spectrum of color shades from the first end of a scale to the second end of the scale. The categorical pain descriptors may be graphical (for example, with a series of facial expressions indicating the increasing degrees of pain). In preferred embodiments, the categorical pain descriptors may include sensory pain descriptors and emotional pain descriptors to provide quantitative measurement of the sensory and emotional dimensions of a subject's pain. For example, each sensory pain descriptor may be assigned a numeric descriptor from a minimum score of 1 to a maximum value of 5. Where a description of a dull or sore pain is assigned as having a score of 1, hurting or pressing is assigned as having a score of 2, radiating or aching as having a score of 3, cramping, burning, searing, crushing as having a score of 4, and splitting, shooting, stabbing, sharp, tearing as having a score of 5. Similarly, emotional pain descriptor may be assigned a numeric descriptor from a minimum score of 1 to a maximum score of 5. Where a description of nagging pain or annoying pain is assigned as having a score of 1, troublesome pain as having a score of 2, tiring, miserable pain as having a score of 3, agonizing, sickening pain as having a score of 4, and killing, unbearable, terrifying, torturing pain as having a score of 5.

In a preferred embodiment, the subject may be asked to use a pain intensity scale to measure the pain intensity at the time of evaluation and report a numeric value. For example, a 0 out of 10 is no pain and a 10 out of 10 is unbearable pain. Various pain scales may be used to measure the intensity of pain. For example, pain scale disclosed in United Kingdom Patent Application Number 0427306.6, WO 03/073937A1, U.S. Pat. No. 5,692,500, U.S. Pat. No. 5,018,526, U.S. Pat. No. 6,258,042 B1, U.S. Design Pat. No. D493,537 S. Other devices that may be useful in the present invention include but are not limited to those discussed in U.S. Pat. No. 5,533,514, U.S. Pat. No. 6,044,303, U.S. Pat. No. 5,941,833.

5.1.2 Aspects of Pain Impact

Pain impacts many aspects of a subject's life, including but not limited to, work activities, daily activities, sports activities, and quality of life. These activities are not mutually exclusive and may overlap. A pain impact scale may be used to quantify the extent to which pain limits these activities and quality of life. Using a pain impact scale, a subject may quantify the amount of impact on activities and translate the amount of impact to a numeric value. For example, a 0 out of 10 is no influence and a 10 out of 10 is major influence to certain activities. These activities may include, for example, work activities, sports activities, and daily activities. Types of work activities include, but are not limited to, for example, physical labour, walking, climbing stairs, sitting at a desk and commuting to work. Sports activities, which may include, but are not limited to, basketball, football, baseball, soccer, golf, racket ball, tennis, swimming, skating, skiing, snowboarding, rollerblading, volleyball, rock climbing, running, jumping, scuba diving, bicycling, walking, yoga, stretching, bowling, etc. Daily activities may include, but are not limited to, various hobbies, gardening, knitting, writing, reading, sitting, typing, carrying heavy bags, shopping, sleeping, talking, eating, physical movement, cleaning, bathing, brushing teeth, cooking, climbing stairs, etc. Quality of life may be determined by the different aspects of well-being of the individual, which may include, but are not limited to, physical well-being, emotional well-being, and social well-being.

5.2 Device for Measuring Pain Impact

The present invention is also directed to an evaluation tool for measuring the amount and/or intensity of pain of a subject and/or pain impact on a subject. The tool is a device comprising a substrate which comprises: (i) a plurality of scales for measuring intensity of pain and/or pain impact as perceived by the subject, each scale comprising a first end and a second end with the first ends of the scales meeting; (ii) an indicator for each scale capable of moving to a position or being located between the two ends of each scale; and (iii) a line which connects the indicators on the plurality of scales.

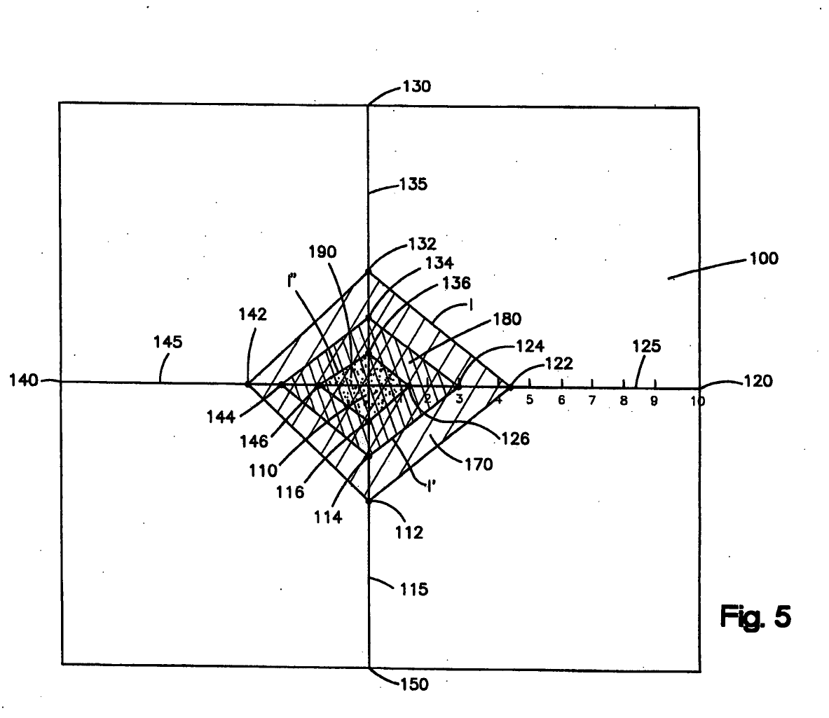

The device comprises a substrate, such as a chart, a card, a paper, a metallic, or a magnetic board, comprising a plurality of scales, or a computer screen on which the chart or plurality of scales is generated by appropriate software. In a specific embodiment, the substrate comprises four scales. FIG. 5 shows a substrate 100 comprising a first scale 125, a second scale 135, a third scale 145, and a fourth scale 115. Each scale comprises a first end 110 and a second end, 120, 130, 140, and 150. The first ends of the scales converge at a single point 110. The angle between each scale may be of any value. In a specific embodiment, the angle between each scale is 90°.

In a preferred embodiment, the first scale measures the amount or intensity of pain. In specific embodiments, the first end has a minimum value and the second end has a maximum value. In specific embodiments, the first end has a value of zero. In specific embodiments, the second end has a value greater than zero. In specific embodiments, the scale is divided into unit measurements. In specific embodiments, the scale is divided evenly into unit measurements of numeric values of ascending order such as 0, 1, 2, 3, 4, 5, 6, 7, 8, 9, 10. In specific embodiments, the scale comprises categorical pain descriptors which may include sensory pain descriptors or emotional pain descriptors. In specific embodiments, the categorical pain descriptors may be graphical or color coded. Exemplary scales that may be used for measuring the intensity of pain are provided in Section 5.1.1.

In specific embodiments, the substrate comprises one or more scales for the measurement of pain impact and/or pain intensity experienced by a subject. In a preferred embodiment, the substrate comprises one scale for the measurement of pain intensity and three scales for the measurement of pain impact. The impact of pain may affect many aspects of a subject's life, including but not limited to, work, daily activities, and quality of life.

The device of the present invention further comprises an indicator that is capable of being positioned between the first end and the second end, inclusively, of each scale. FIG. 5 shows that a first indicator 122 between the first end 110 and the second end 120 on the first scale 125; a second indicator 132 between the first end 110 and the second end 130 on the second scale 135; a third indicator 142 between the first end 110 and the second end 140 on the third scale 145; and a fourth indicator 112 between the first end 110 and the second end 150 on the fourth scale 115. In preferred embodiments, the indicator is a thumb-nail, a magnet, an adhesive marker, or a marking drawn on the scale.

In specific embodiments, the first scale is a pain intensity scale, the second scale is a pain impact scale to quantify the extent to which the pain limits professional activities, the third scale is a pain impact scale to quantify the extent to which the pain limits daily activities, the fourth scale is a pain impact scale to quantify the extent to which the pain limits quality of life. Regarding the second pain impact scale that may be used for measuring pain impact on work, for example, a 0 out of 10 is no influence and a 10 out of 10 is major influence on professional activities. The third pain impact scale is used to quantify the extent to which the pain limits daily activities. For example, a 0 out of 10 is no influence and a 10 out of 10 is major influence on daily activities. The fourth pain impact scale is to quantify the extent to which the pain limits quality of life, including but not limited to social activities, quality of sleep, moments of pleasure. For example, a 0 out of 10 is no influence and a 10 out of 10 is major influence on quality of life. In specific embodiments, the indicator is capable of being moved to or located at a position that represents any value on the scale.

The device of the present invention may further comprise a flexible line, such as but not limited to, a string, a wire, or an elastic band, that is capable of connecting the indicators on the scales. In another embodiment, the indicators on the scales are connected by a line which is drawn or infilled on the substrate. In FIG. 5, first indicators 122, 132, 142, and 112 are connected with a first line, 1, such that the first lines and scales 125, 135, 145, and 115 enclose a first surface area 170 having a first size. The first size represents a first measurement of pain impact at a certain pain intensity at a first time point. In specific embodiments, second indicators 124, 134, 144, 114 are connected with a second line, 1′, such that the second lines and scales 125, 135, 145, and 115 enclose a second surface area 180 having a second size. The second size represents a second measurement of pain impact at a certain pain intensity at a second time point. In specific embodiments, third indicators 126, 136, 146, 116 are connected with a third line, 1″, such that the third lines and scales 125, 135, 145, and 115 enclose a third surface area 190 having a third size. The third size represents a third measurement of pain impact at a certain pain intensity at a third time point.

Examples of therapeutic agents that may be used for the treatment of patients suffering from pain include, but are not limited to, an opioid agonist, a non-opioid analgesic, a non-steroid anti-inflammatory agent, an antimigraine agent, a Cox-II inhibitor, an antiemetic, an anticonvulsant, an antidepressant, a Ca2+-channel blocker, an anticancer agent, an anti-anxiety agent or a combination of any of the foregoing.

### EQUIVALENTS

The present invention is not to be limited in scope by the specific embodiments described which are intended as illustrations of individual aspects of the invention, and functionally equivalent methods and components are within the scope of the invention. Indeed, various modifications of the invention, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference into the specification to the same extent as if each individual publication, patent or patent application was specifically and individually indicated to be incorporated herein by reference.

Citation or discussion of a reference herein shall not be construed as an admission that such is prior art to the present invention.

What is claimed is:

1. A method to measure the level of pain impact and/or pain intensity of a subject comprising the steps of:
(a) providing a substrate comprising a plurality of scales, each of the scales comprising a first end and a second end, wherein the first ends of the scales converge at a single point;
(b) marking a first point at a position between the first end and the second end inclusively on each scale as determined by the subject; and
(c) connecting each pair of points on adjacent scales with a first line in which the first lines and the scales enclose a first surface area having a first size.

2. The method of claim 1 wherein the plurality of scales comprises a first scale, a second scale, a third scale and a fourth scale.

3. The method of claim 2 wherein the first end of the first scale has a minimum value and the second end of the first scale has a maximum value.

4. The method of claim 2 wherein the first scale comprises a spectrum of color shades which vary from the first end to the second end.

5. The method of claim 2 wherein the first scale measures the intensity of pain.

6. The method of claim 2 wherein the second scale measures the level of pain impact on quality of life.

7. The method of claim 2 wherein the third scale measures the level of pain impact on work activities.

8. The method of claim 2 wherein the fourth scale measures the level of pain impact on daily activities.

9. The method of claim 2 wherein the substrate further comprises a fifth scale.

10. The method of claim 5 wherein the first scale comprises graduated intervals.

11. The method of claim 5 wherein the position of the marking on the first scale is determined according to the intensity of pain experienced by the subject.

12. The method of claim 6 wherein the position of the marking on the second scale is determined according to the pain impact on quality of life experienced by the subject.

13. The method of claim 7 wherein the position of the marking on the third scale is determined according to the pain impact on work activities experienced by the subject.

14. The method of claim 8 wherein the position of the marking on the fourth scale is determined according to the pain impact on daily activities experienced by the subject.

15. The method of claim 1 wherein the position of the marking of the first point is determined during a first time point.

16. The method of claim 1 wherein the position of the marking of the first point is determined using a device for measuring pain.

17. The method of claim 1 further comprising the steps of:
(d) marking a second point between the first end and the second end inclusively on each scale; and
(e) connecting each pair of second points on adjacent scales with a second line in which the second lines and the scales enclose a second surface area having a second size.

18. The method of claim 17 wherein the position of the marking of the second point is determined during a second time point.

19. The method of claim 17 wherein the position of the marking of the second point occurs after administration of a first treatment.

20. The method of claim 17 further comprising the step of: (f) comparing the first size of the first surface area and the second size of the second surface area, wherein a decrease in the second size of the second surface area compared to the first size of the first surface area indicates a decrease in pain impact and/or pain intensity, and wherein an increase in the second size of the second surface area compared to the first size of the first surface area indicates an increase in pain impact and/or pain intensity.

21. The method of claim 17 further comprising the steps of:
(f) marking a third point between the first end and the second end inclusively on each scale; and
(g) connecting each pair of third points on adjacent scales with a third line in which the third lines and the scales enclose a third surface area having a third size.

22. The method of claim 19 wherein the treatment is an administration of an opioid agonist, a non-opioid analgesic, a non-steroid anti-inflammatory agent, an antimigraine agent, a Cox-II inhibitor, an antiemetic, an anticonvulsant, an antidepressant, a Ca²⁺-channel blocker, an anticancer agent, an anti-anxiety agent or any combination of two or more of the foregoing.

23. The method of claim 21 wherein the position of the marking of the third point occurs after administration of a second treatment.

24. The method of claim 21 further comprising the step of:
(h) comparing the second size of the second surface area and the third size of the third surface area, wherein a decrease in the third size of the third surface area compared to the second size of the second surface area indicates a decrease in pain impact and/or pain intensity, and wherein an increase in the second size of the second surface area compared to the first size of the first surface area indicates an increase in pain impact and/or pain intensity.
